(19) United States
(12) Patent Application Publication
(10) Pub. No.: US 2022/0330684 A1
(43) Pub. Date: Oct. 20, 2022

(54) Portable Carrying Case Assembly

(71) Applicant: Eduardo Torres, Sanger, CA (US)

(72) Inventor: Eduardo Torres, Sanger, CA (US)

(21) Appl. No.: 17/233,708

(22) Filed: Apr. 19, 2021

(51) Int. Cl. A45F 5/06; A61B 42/40; B65D 83/08

(52) U.S. Cl. CPC A45F 5/06; A61B 42/40; B65D 83/0805; A45F 2200/05

(57) ABSTRACT

A portable carrying case assembly for carrying a box of disposable gloves includes a container that has a plurality of intersecting sides such that the container has a rectilinear shape for containing a box of disposable gloves. A cover is positionable on the container for closing the container. The cover has an opening extending through the cover to facilitate the disposable gloves to be drawn outwardly therefrom when the cover is positioned on the container. A clip is coupled to the container to engage an article of clothing for carrying the container in a hands free manner.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to carrying case devices and more particularly pertains to a new carrying case device for carrying a box of disposable gloves. The device includes a rectilinear container, a cover with a dispensing slot that is positionable on the container and a clip coupled to the container for engaging an article of clothing.

### (2) Description of Related Art Including Information Disclosed Under 37 CFR 1.97 and 1.98

The prior art relates to carrying case devices, including a top loading hard case for carrying disposable gloves. The prior art discloses a variety of soft cases for carrying disposable gloves. The prior art discloses a method of loading disposable gloves into a box. In no instance does the prior art disclose a case for carrying disposable gloves that includes a cover positioned on a front side of the case.

### BRIEF SUMMARY OF THE INVENTION

An embodiment of the disclosure meets the needs presented above by generally comprising a container that has a plurality of intersecting sides such that the container has a rectilinear shape for containing a box of disposable gloves. A cover is positionable on the container for closing the container. The cover has an opening extending through the cover to facilitate the disposable gloves to be drawn outwardly therefrom when the cover is positioned on the container. A clip is coupled to the container to engage an article of clothing for carrying the container in a hands free manner.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a portable carrying case assembly according to an embodiment of the disclosure.

FIG. 2 is a back perspective view of an embodiment of the disclosure.

FIG. 3 is a front view of an embodiment of the disclosure.

FIG. 4 is a cross sectional view taken along line 4-4 of FIG. 3 of an embodiment of the disclosure.

FIG. 5 is an exploded perspective view of an embodiment of the disclosure.

FIG. 6 is a perspective in-use view of an embodiment of the disclosure.

### DETAILED DESCRIPTION OF THE INVENTION

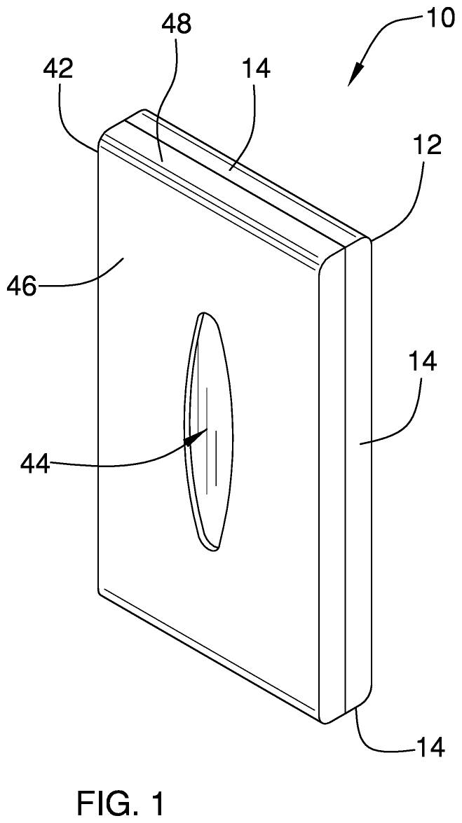

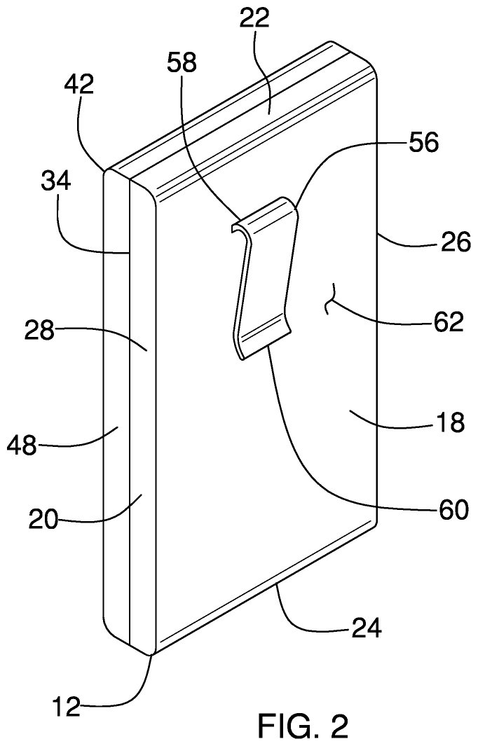

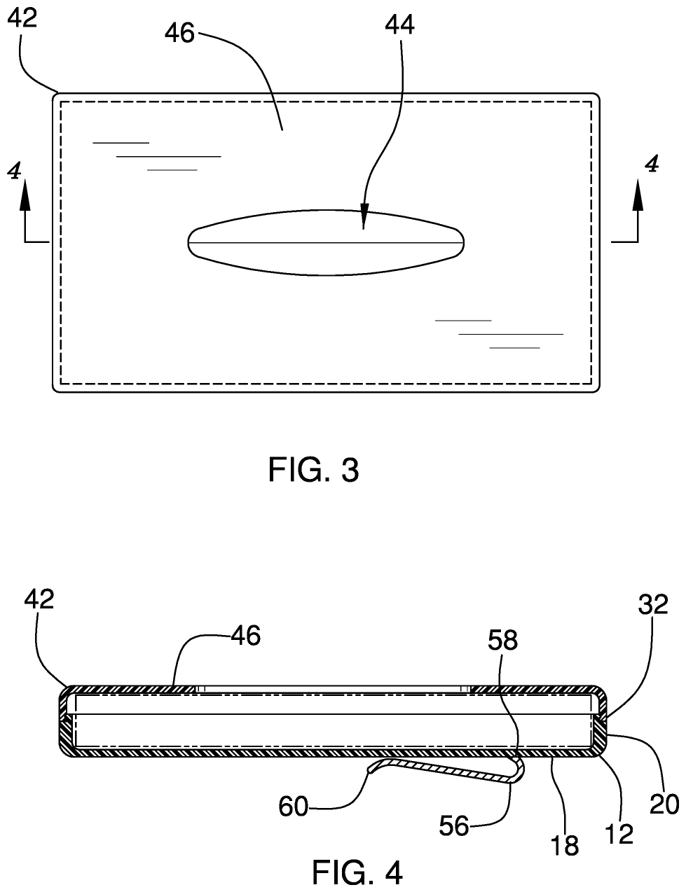

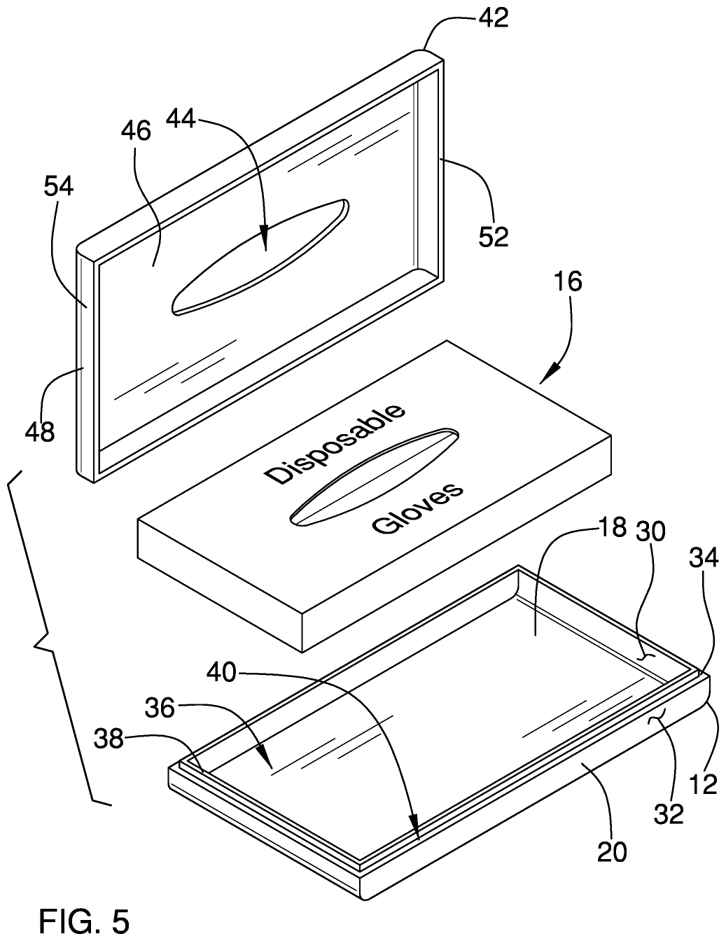

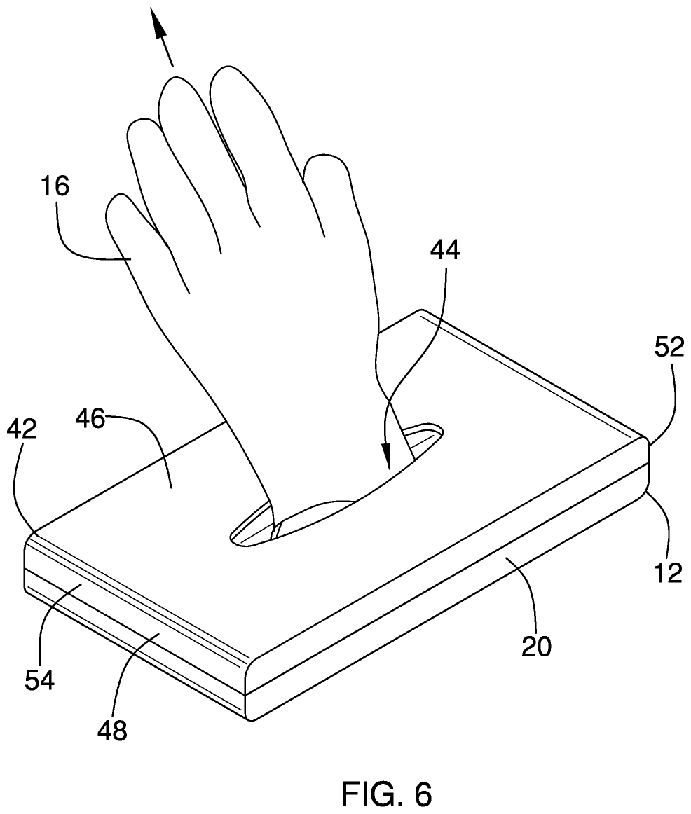

With reference now to the drawings, and in particular to FIGS. 1 through 6 thereof, a new carrying case device embodying the principles and concepts of an embodiment of the disclosure and generally designated by the reference numeral 10 will be described.

As best illustrated in FIGS. 1 through 6, the portable carrying case assembly 10 generally comprises a container 12 that has a plurality of intersecting sides 14 such that the container 12 has a rectilinear shape to contain a box of disposable gloves 16. The disposable gloves 16 may be rubber gloves, nitrile gloves or any other type of disposable gloves worn in a medical environment, for example. The container 12 has a rear wall 18 and a perimeter wall 20 extending away therefrom, and the perimeter wall 20 has a top side 22, a bottom side 24, a first lateral side 26 and a second lateral side 28. The container 12 is elongated between the top side 22 and the bottom side 24, and the perimeter wall 20 has an inwardly facing surface 30, an outwardly facing surface 32 and a distal edge 34 with respect to the rear wall 18.

The distal edge 34 defines an opening 36 into the container 12 and the distal edge 34 has a lip 38 extending away from the distal edge 34. The lip 38 extends around each of the top side 22, the bottom side 24, the first lateral side 26 and the second lateral side 28. The lip 38 is aligned with the inwardly facing surface 30 of the perimeter wall 20 to define an engagement portion 40 of the distal edge 34 extending between the lip 38 and the outwardly facing surface 32 of the perimeter wall 20. An intersection between each of the top side 22, the bottom side 24 and the rear wall 18 is rounded.

A cover 42 is provided and the cover 42 is positionable on the container 12 for closing the container 12. The cover 42 has an opening 44 extending through the cover 42 to facilitate the disposable gloves 16 to be drawn outwardly therefrom when the cover 42 is positioned on the container 12. The cover 42 has a forward wall 46 and an outer wall 48 extending away therefrom, and the outer wall 48 has an upper side 50, a lower side 52 and a distal edge 54 with respect to the forward wall 46. The cover 42 is elongated between the upper side 50 and the lower side 52, and the opening 44 extends through the forward wall 46. The opening 44 is elongated between the upper side 50 and the lower side 52 such that the opening 44 has an ovoid shape, and the opening 44 is centrally positioned on the forward wall 46. The distal edge 54 of the outer wall 48 abuts the engagement portion 40 of the distal edge 34 of the perimeter wall 20 of the container 12 when the cover 42 is positioned on the container 12. An intersection between the upper side 50, the lower side 52 and the forward wall 46 is rounded.

A clip 56 is coupled to the container 12 to engage an article of clothing, such as a waist band or a waist belt, for carrying the container 12 in a hands free manner. The clip 56 has a first end 58 and a second end 60, and the clip 56 undulates between the first end 58 and the second end 60. The first end 58 is coupled to a rear surface 62 of the rear wall 18 of the container 12 and the clip 56 is biased to rest against the rear surface 62. In this way the clip 56 can compress the article of clothing between the clip 56 and the rear surface 62 of the rear wall 18.

In use, the box of disposable gloves 16 is positioned in the container 12 and the cover 42 is positioned on the container 12. The clip 56 is worn on the article of clothing thereby facilitating the box of disposable gloves 16 to be carried in a hands free manner. In this way a user, such as a medical professional, has continual access to the disposable gloves 16 during the course of duty. Thus, the user can more efficiently practice cleanliness habits in a medical environment, for example.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A portable carrying case assembly for transporting a plurality of latex gloves with a user, said assembly comprising:
a container having a plurality of intersecting sides such that said container has a rectilinear shape wherein said container is configured to contain a box of disposable gloves;
a cover being positionable on a front side of said container for closing said container, said cover having an opening extending through said cover wherein said opening is configured to facilitate the disposable gloves to be drawn outwardly therefrom when said cover is positioned on said container; and
a clip being coupled to said container wherein said clip is configured to engage an article of clothing for carrying said container in a hands free manner.

2. The assembly according to claim 1, wherein said container has a rear wall and a perimeter wall extending away therefrom, said perimeter wall having a top side, a bottom side, a first lateral side and a second lateral side, said container being elongated between said top side and said bottom side, said perimeter wall having an inwardly facing surface, an outwardly facing surface and a distal edge with respect to said rear wall, said distal edge defining an opening into said container.

3. The assembly according to claim 2, wherein said distal edge has a lip extending away from said distal edge, said lip extending around each of said top side, said bottom side, said first lateral side and said second lateral side, said lip being aligned with said inwardly facing edge of said perimeter wall to define an engagement portion of said distal edge extending between said lip and said outwardly facing surface of said perimeter wall, an intersection between each of said top side, said bottom side and said rear wall being rounded.

4. The assembly according to claim 2, wherein said clip has a first end and a second end, said clip undulating between said first end and said second end, said first end being coupled to a rear surface of said rear wall of said container, said clip being biased to rest against said rear surface wherein said clip is configured to compress the article of clothing between said clip and said rear surface of said rear wall.

5. A portable carrying case assembly for transporting a plurality of latex gloves with a user, said assembly comprising:
a container having a plurality of intersecting sides such that said container has a rectilinear shape wherein said container is configured to contain a box of disposable gloves, said container having a rear wall and a perimeter wall extending away therefrom, said perimeter wall having a top side, a bottom side, a first lateral side and a second lateral side, said container being elongated between said top side and said bottom side, said perimeter wall having an inwardly facing surface, an outwardly facing surface and a distal edge with respect to said rear wall, said distal edge defining an opening into said container, said distal edge having a lip extending away from said distal edge, said lip extending around each of said top side, said bottom side, said first lateral side and said second lateral side, said lip being aligned with said inwardly facing edge of said perimeter wall to define an engagement portion of said distal edge extending between said lip and said outwardly facing surface of said perimeter wall, an intersection between each of said top side, said bottom side and said rear wall being rounded;
a cover being positionable on a front side of said container for closing said container, said cover having an opening extending through said cover wherein said opening is configured to facilitate the disposable gloves to be drawn outwardly therefrom when said cover is positioned on said container, said cover having a forward wall and an outer wall extending away therefrom, said outer wall having an upper side, a lower side and a distal edge with respect to said forward wall, said cover being elongated between said upper side and said lower side, said opening extending through said forward wall, said opening being elongated between said upper side and said lower side such that said opening has an ovoid shape, said opening being centrally positioned on said forward wall, said distal edge of said outer wall abutting said engagement portion of said distal edge of said perimeter wall of said container when said cover is positioned on said container, an intersection between said upper side, said lower side and said forward wall being rounded; and
a clip being coupled to said container wherein said clip is configured to engage an article of clothing for carrying said container in a hands free manner, said clip having a first end and a second end, said clip undulating between said first end and said second end, said first end being coupled to a rear surface of said rear wall of said container, said clip being biased to rest against said rear surface wherein said clip is configured to compress the article of clothing between said clip and said rear surface of said rear wall.

5. A portable carrying case system for transporting a plurality of latex gloves with a user, said system comprising:
a box of disposable gloves;
a container having a plurality of intersecting sides such that said container has a rectilinear shape thereby facilitating said container to contain said box of disposable gloves, said container having a rear wall and a perimeter wall extending away therefrom, said perimeter wall having a top side, a bottom side, a first lateral side and a second lateral side, said container being elongated between said top side and said bottom side, said perimeter wall having an inwardly facing surface, an outwardly facing surface and a distal edge with respect to said rear wall, said distal edge defining an opening into said container, said distal edge having a lip extending away from said distal edge, said lip extending around each of said top side, said bottom side, said first lateral side and said second lateral side, said lip being aligned with said inwardly facing edge of said perimeter wall to define an engagement portion of said distal edge extending between said lip and said outwardly facing surface of said perimeter wall, an intersection between each of said top side, said bottom side and said rear wall being rounded;
a cover being positionable on said container for closing said container, said cover having an opening extending through said cover to facilitate each of said disposable gloves to be drawn outwardly therefrom when said cover is positioned on said container, said cover having a forward wall and an outer wall extending away therefrom, said outer wall having an upper side, a lower side and a distal edge with respect to said forward wall, said cover being elongated between said upper side and said lower side, said opening extending through said forward wall, said opening being elongated between said upper side and said lower side such that said opening has an ovoid shape, said opening being centrally positioned on said forward wall, said distal edge of said outer wall abutting said engagement portion of said distal edge of said perimeter wall of said container when said cover is positioned on said container, an intersection between said upper side, said lower side and said forward wall being rounded; and
a clip being coupled to said container wherein said clip is configured to engage an article of clothing for carrying said container in a hands free manner, said clip having a first end and a second end, said clip undulating between said first end and said second end, said first end being coupled to a rear surface of said rear wall of said container, said clip being biased to rest against said rear surface wherein said clip is configured to compress the article of clothing between said clip and said rear surface of said rear wall.
